(19) United States
(12) Patent Application Publication
(10) Pub. No.: US 2006/0104939 A1
(43) Pub. Date: May 18, 2006

(54) Degrading lignocellulosic materials

(76) Inventors: Anthony Dale Covington, Northampton (GB); Christine Stella Evans, London (GB); Millie Ullah, London (GB)

Correspondence Address: FISH & RICHARDSON PC, P.O. BOX 1022, MINNEAPOLIS, MN, 55440-1022, US

(21) Appl. No.: 10/523,054

(22) PCT Filed: Aug. 4, 2003
(86) PCT No.: PCT/GB03/03384

§ 371 Date: Oct. 27, 2005

(30) Foreign Application Priority Data
Aug. 2, 2002 (GB) 0218001.6

(51) Int. Cl. A61L 9/01; A61L 9/00

(52) U.S. Cl. 424/76.1; 435/262

(57) ABSTRACT

A method for the degradation of lignocellulosic material by applying to the material an enzyme composition which is a mixture comprising at least a cellulase, xylanase and ligninase, and optionally other enzymes, such as a protease, lipase, urease, uricase, and/or pectinase, to solubilise or decompose the material at least partially. The method may be used for removing a biological deposit from a surface or location on or in which it is undesirably deposited. Typical deposits include human or animal faeces, bird droppings, and leaves. The cellulase, xylanase and ligninase component may be obtained as a mixture by cultivating a White Rot fungus, preferably using cattle dung, or a liquid extract, as an inducer.

This invention is concerned with degrading lignocellulosic materials. The invention is especially suitable for cleaning biological deposits, such as animal faeces, from surfaces where the deposits cause inter alia problems of hygiene (such as dog faeces on pavements), appearance (such as bird droppings on buildings), or safety (such as wet leaves on roads or railways). In particular, it is concerned with the production of enzyme mixtures, specifically designed to degrade the deposits.

Dung on cattle creates problems for hygiene on the dairy farm and more particularly at the abattoir, where there is risk of contaminating the carcase with faecal organisms, notably including E. coli O0157. Typically this is not addressed by the respective industries, creating a residual problem that must be addressed by the global leather industry, particularly in respect of beef cattle which form the biggest source of hides for the leather industry.

Dung must be removed from the hides in the early stages of processing, as part of the cleansing operations, leading to tanning and the production of high quality leather. Removal of dung is difficult; the composite material created by hair and dry dung is resistant to solubilisation, even in the presence of surfactants. It is accepted in the industry that even the enzymes offered as soaking auxiliaries do not have any useful effect in this regard: those enzymes include proteases, lipases and amylases, but no claims are made by the supply houses for any positive effect on dung. In studies of the effects of enzymes on the solubilisation of dry dung, it was confirmed that those types of soaking enzymes are ineffective (Enzymatic removal of dung from hides. N. Auer, A. D. Covington, A. S. Evans, M. Natt, M. Tozan; J. Soc. Leather Technol. Chem., 83(4), 215, 1999.). Therefore, tanners are obliged to risk bacterial damage in prolonged soaking, or to remove the dung with hair, incurring additional chemical cost and limiting the options for disposing of the contaminated hair.

In GB 2,325,241 it is demonstrated that dung is removed efficiently and effectively from animal skins intended for leather production, or even from the skin of live animals, by targeting the main components of the dung with specifically acting enzymes. It is disclosed that the lignocellulosic material in dung, from partially degraded plant cell walls, can be solubilised with an enzyme composition containing at least one of cellulase, xylanase and ligninase, preferably a mixture of cellulase and xylanase, optionally containing ligninase if available.

Subsequent unpublished studies by the present inventors have showed that the lignocellulosic material is solubilised as the complex, rather than by selective damage of one or two of the constituents. This indicates that it is necessary simultaneously to degrade the lignin which surrounds the fibres, to expose the cellulose to attack, and to break the hemicellulose linkages between the cellulose chains, in order to dissolve the complex. The present inventors′ solution to the problem of dung in the tannery is to apply a treatment of the three enzymes, cellulase, xylanase and ligninase, at a suitable activity ratio obtainable in a single cultivation step from white rot fungi which is the subject-matter of our copending patent application no. PCT/GB2003/00113.

The present invention is based on the appreciation that similarly tailored mixtures of enzymes can be used to remove biological deposits from surfaces other than animal skins and other locations, where such deposits result in issues of inter alia hygiene, appearance and safety.

In its broadest aspect the present invention provides a method for the degradation of lignocellulosic material by applying to the material an enzyme composition which is a mixture comprising at least a cellulase, xylanase and ligninase, and optionally other enzymes, to solubilise or decompose the material at least partially.

In a particular aspect the present invention provides a method of removing a biological deposit from a surface or location on or in which it is undesirably deposited, by applying to the deposit an enzyme composition which is a mixture comprising a cellulase, xylanase and ligninase, and optionally other enzymes, to solubilise or decompose the deposit at least partially.

Other enzymes that may be included in the mixtures used in this invention may selected from, for example, a protease, lipase, urease, uricase, and pectinase.

The enzyme mixtures used in the present invention may be formed by blending individual enzymes as disclosed in GB 2,325,241, the contents of which are incorporated herein by reference. Also further enzymes may be added to address the specific components of the deposit.

For example, while a mixture of the three enzymes cellulase, xylanase and ligninase is effective for cattle dung, for other animal faeces additional enzymes such as uricases and ureases may be needed for effective solubilisation or decomposition. For other biological deposits, it may be desirable to add proteases or lipases.

More specifically, but without limitation, for removal of dog and fox faeces an enzyme mixture of protease, lipase, urease, cellulase, xylanase and ligninase is proposed; for bird droppings a mixture of uricase, cellulase, xylanase and ligninase is proposed; for leaves and compost acceleration a mixture of pectinase, cellulase, xylanase and ligninase is proposed; for chemical toilets for humans or discharge from train toilets onto railway lines a mixture of protease, lipase, urease, cellulase, xylanase and ligninase is proposed.

However, while blends of individual enzymes are suitable for small scale use, this is not practical in commercial practice, because although cellulase and xylanase and other the other listed enzymes are available in commercial quantities, there is at present no commercial source of ligninase. Therefore, there is a need to produce the ligninase by large scale fermentation of a suitable microorganism. In this context the present inventors have sought to create the conditions which would force a microorganism to express at least the core mixture of three enzymes required for removal of deposits.

The present inventors have made the unexpected finding that white rot fungi can be induced to produce a mixture of the enzymes cellulase, xylanase and ligninase which contains ligninase (laccase) in a sufficient quantity and appropriate ratios to degrade lignocellulosic materials, for example as found in undesirable biological deposits.

The core enzyme mixture for removing biological deposits from surfaces can be prepared by cultivating a fungus selected from the class of White Rot Fungi in a liquid growth medium and harvesting the enzymes produced by the fungus from the liquid growth medium.

Suitable white rot fungi are found (but not exclusively) in the family Polyporaceae. Especially suitable are fungi of the species Coriolus, Pleurotus, and Ganoderma, in particular Coriolus versicolor (also known as Trametes versicolor), Pleurotus ostreatus and Ganoderma applanatum. Other suitable white rot fungi can easily be determined by routine testing for ability to produce all three enzymes, rate of growth, levels of enzyme activities etc.

Some white rot fungi decompose lignin by production of a peroxidase, (which require additionally hydrogen peroxide) rather than laccase. A typical example is the species Phanerochaete, especially Phanerochaete chrysosporium. These white rot fungi are within the scope of the present invention, but the resultant enzyme mixtures are less preferable for the treatment of animal skins because of the need to provide a co-substrate (hydrogen peroxide) for the peroxidase to act on.

The present inventors have found that white rot fungi that produce a mixture of cellulase, xylanase and laccase typically do not produce laccase in sufficient quantities for optimum treatment of biological deposits. However they have discovered that this problem can be overcome by cultivating the fungus in the presence of a suitable inducer. Advantageously the inducer to promote production of enzymes is cattle dung, preferably in sterile form, as a powder or liquid extract, especially an aqueous extract.

Handling of the inducer and the accuracy of measurement is improved by use of a liquid extract of dung as the inducer. Accordingly as a further aspect the present invention provides a method of preparing an enzyme mixture suitable for cleaning animal skins and degrading lignocellulosic materials and biological deposits which comprises cultivating a fungus selected from the class of White Rot Fungi in a liquid growth medium in the presence of a liquid extract of animal faeces, especially an aqueous extract of cattle dung, as an inducer, and harvesting the enzymes produced by the fungus from the liquid growth medium.

In tests carried out by the present inventors on the fungi Coriolus versicolor, Pleurotus ostreatus and Ganoderma applanatum, Coriolus versicolor and Pleurotus ostreatus were the fastest growing species, covering a 7 cm malt-agar Petri plate with hyphae from a central inoculum within six days, whereas Ganoderma applanatum, took twelve days.

C. versicolor and P. ostreatus produced similar amounts of cellulase and xylanase in the liquid media with cellulose or xylan as substrates over a ten day growth period, but differed in their production of laccase. P. ostreatus produced only low levels of laccase over ten days, with most laccase produced after growing for twenty days or more, when cellulase and xylanase activities had diminished considerably. Laccase activity was not increased significantly in the presence of a lignin mimic inducer in the first ten days of culture. In contrast, laccase production by C. versicolor doubled in the presence of an inducer compound, with the highest amount of laccase produced by any organism after eight days growth.

Surprisingly, it was found that the ratios of the three enzyme activities required to treat dung, and also found to be effective against other biological deposits, especially faeces, containing lignocellulosic materials, could be controlled by the nature of the growing medium. In particular, the difficulty of producing enough ligninase (laccase) could be overcome by adding a growth medium auxiliary as an inducer. Thus, the required enzyme mixture can be produced in a single fermentation step.

Unexpectedly, it was found that the inclusion of cattle dung, most suitably sterilised before use, significantly broadened the peak of laccase production. This is of great value in the context of commercial production, since it greatly assists in the ability to harvest a suitably proportioned enzyme mixture.

In the present invention, the fungi are suitably cultivated in a liquid nutrient medium with a nitrogen source and a carbon source, and preferably an inducer in the form of sterile dung or an aqueous dung extract. After a suitable period of growth, fungal growth is removed and enzymes in the culture fluid are harvested.

Suitably the fungi are added to the nutrient medium in pelletised form, to assist in subsequent removal by filtration, together with any dung residue. The filtrate containing the enzymes is preferably concentrated, for example using a membrane concentrator with a cut off at 10,000 Daltons. Then the concentrate is preferably dried. Freeze drying will provide the desired enzymes as a lyophilised powder. Spray drying or other drying may also be used.

The powder may be stored or packaged for future use as a cleaning composition. The enzyme powder may be mixed with an inert bulking agent, so that technicians are able to weigh out enzyme dosages in, for example 100 gm units rather than gram units. Alternatively the enzyme mixture, or bulked mixture, may be pre-packaged in unit doses. The bulking agent is suitably selected so that it will not leave a residue on the treated surface. Sodium chloride may be used.

Alternatively, a lyophilised powder may be reconstituted with water, to provide the user with a liquid concentrate.

Whether in either powder or liquid form as supplied to the cleaning personnel, the enzyme composition is preferably applied to the deposit as an aqueous solution or dispersion, optionally formulated with thickening agents to prevent unnecessary spread of the formulation, or with surfactants to assist in the cleaning process.

In a further aspect the present invention provides as enzyme composition useful to solubilise or decompose a biological deposit, which is a enzyme mixture comprising at least a cellulase, xylanase and ligninase, and at least one other enzyme selected from a protease, lipase, urease, uricase, and pectinase.

Preferably the cellulase, xylanase and ligninase component is obtained by cultivating a White Rot in a liquid growth medium and harvesting the enzymes produced by the fungus from the liquid growth medium. Most preferably the fungus is cultivated in the presence of dung or a dung extract as an auxiliary growth medium or inducer.

The invention is further illustrated by the following Examples and by reference to the accompanying drawings, in which:

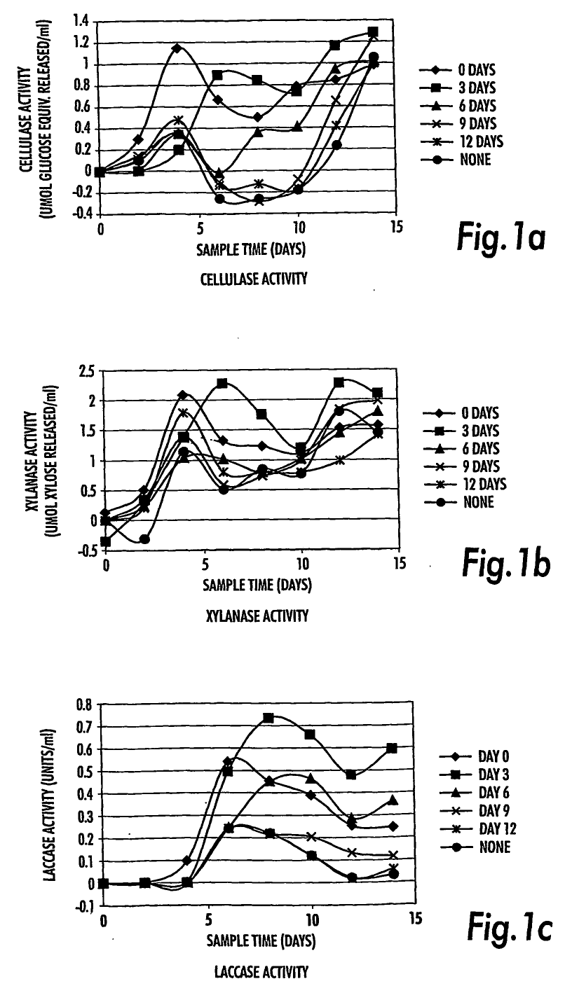

FIGS. 1 a, 1 b and 1 c show production of enzymes after adding 1% (w/v) inducer after 3, 6, 9 or 12 days into cultures of C. versicolor containing 2% (w/v); carboxymethyl cellulose (CMC) as carbon source;

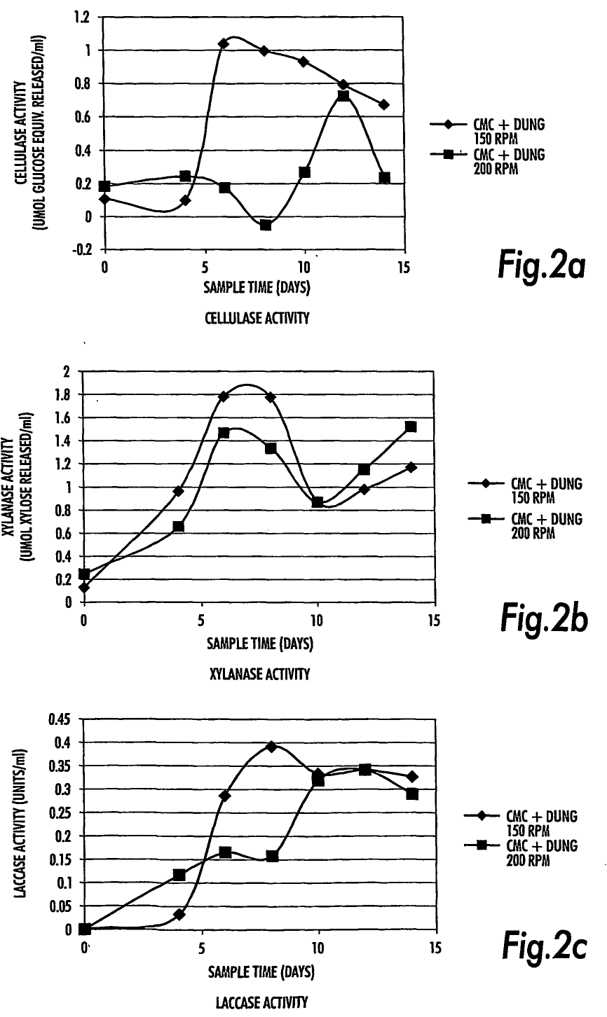

FIGS. 2 a, 2 b and 2 c shows a comparison of shaker speeds in enzyme production.

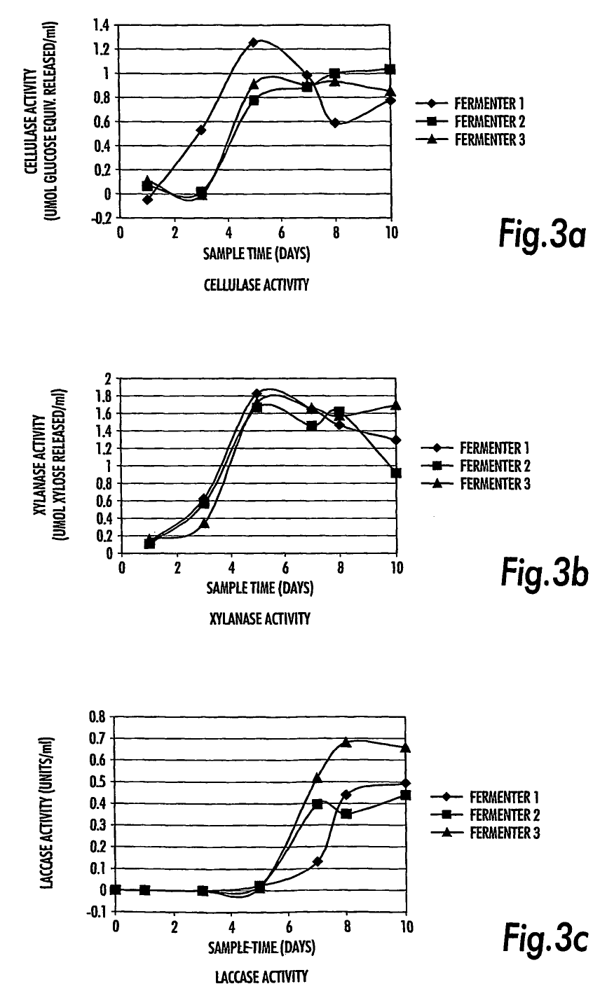

FIGS. 3 a, 3 b and 3 c show further results from fermentation of C. versicolor on the 2 litre scale;

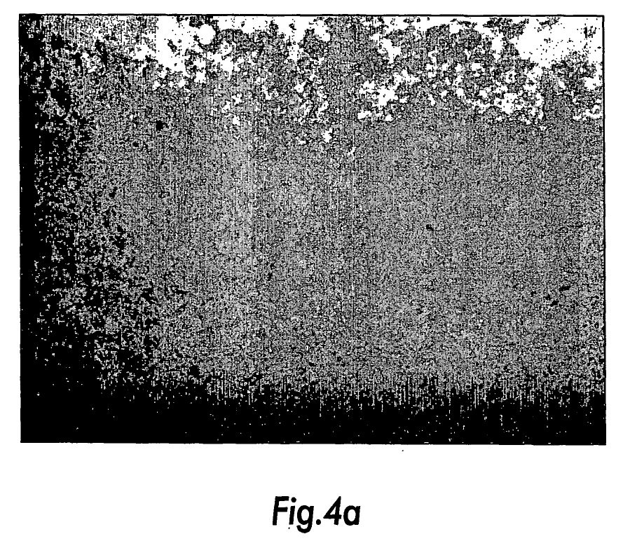

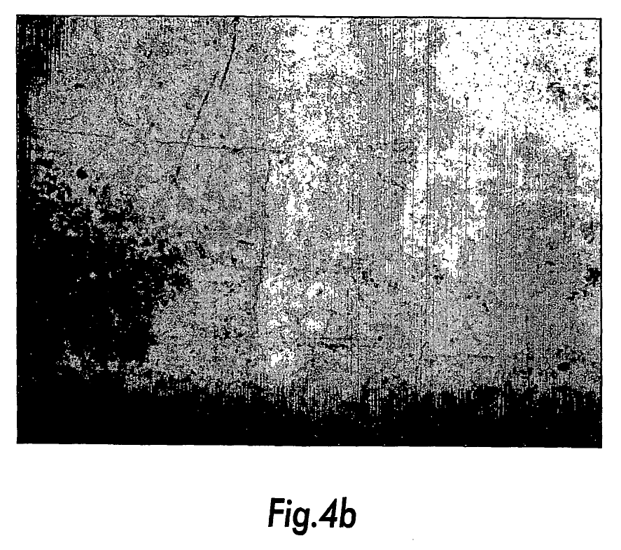

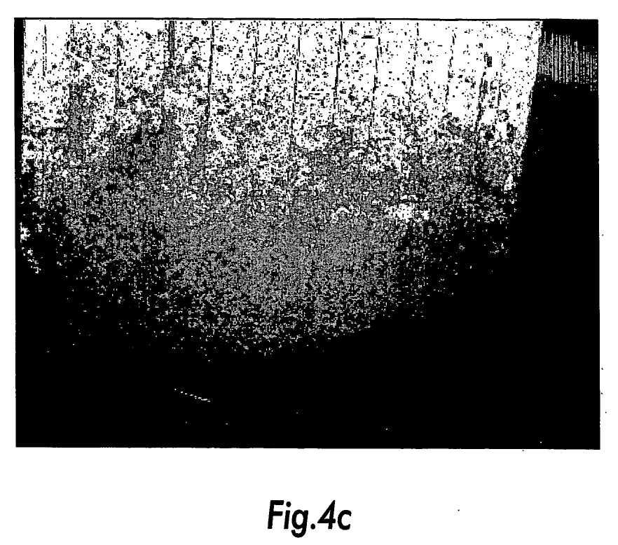

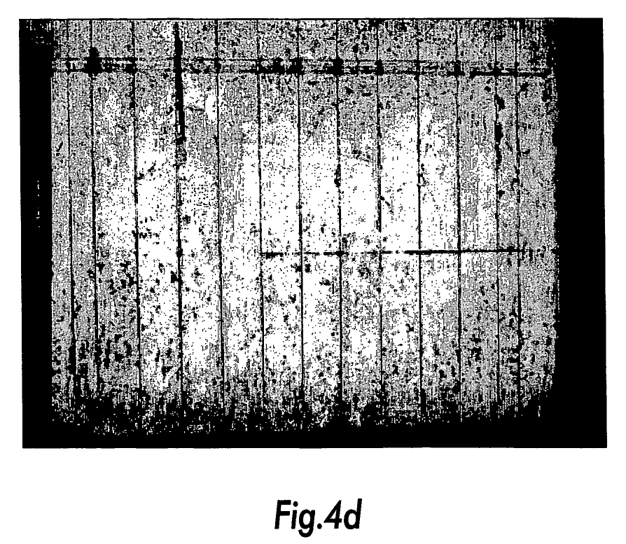

FIGS. 4 a and 4 b show the effects of the cleaning process of the invention on a concrete wall before treatment (4 a) and after treatment (4 b);

FIGS. 4 c and 4 d show the effects of the cleaning process of the invention on a painted door before treatment (4 c) and after treatment (4 d);

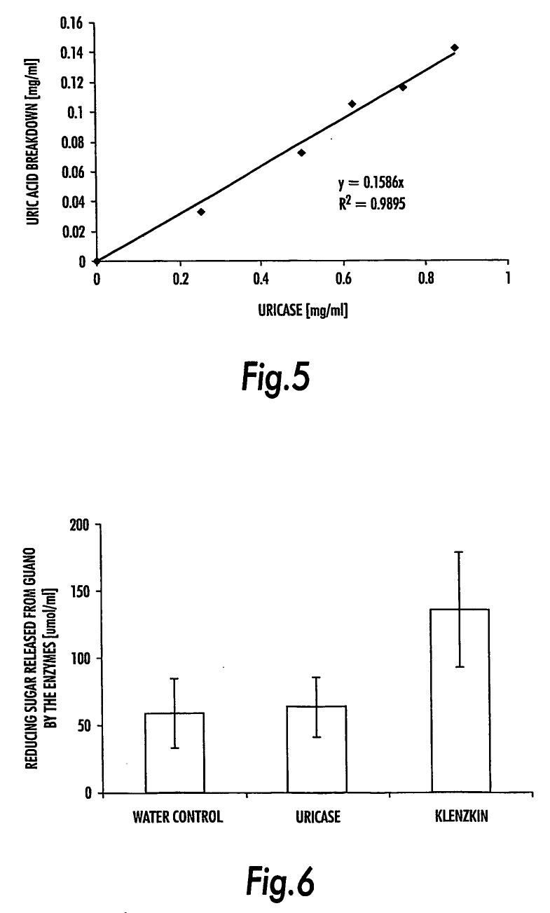

FIG. 5 shows the activity of uricase on pigeon guano; and

FIG. 6 shows the release of reducing sugars from pigeon guano after treatment with various enzymes.

### EXAMPLE 1

### Induced Enzyme Production by C. versicolor in Shake Flask Cultures

The liquid growth media in these trials were based on a mineral salts medium with ammonium nitrate as nitrogen source (see—E. Abrams; National Bureau of Standards Misc. Publications no. 188. U.S. Dept. of Commerce, Washington) and included carboxymethyl cellulose (CMC) as carbon source. The inventors′ proposed inducer for laccase, dried sterilised cow dung, was added at different time intervals during fungal growth. The results are shown graphically in the appended FIGS. 1a, 1b and 1c which show the effects on the production of enzymes of adding 1% (w/v) inducer after 3, 6, 9 or 12 days into cultures of C. versicolor containing 2% (w/v) CMC as carbon source.

For laccase production, addition of the inducer after three days of fungal growth gave the highest yield of laccase in cultures at day 8 of growth. For cellulase and xylanase activities, addition of the inducer after three days also yielded the highest enzymes activities, but from day 6 onwards.

TABLE I
The effect of inducer on the production of laccase.

| | Inducer offered (% w/v) | | | | |
|---|---|---|---|---|---|
| | 0 | 0.1 | 0.2 | 0.5 | 1.0 |
| Laccase activity at day 8 | 0.2 | 0.22 | 0.37 | 0.62 | 0.59 |
| (ΔOD₄₄₀/ml/min) | | | | | |

### EXAMPLE 2

### The Role of Carbon Source in Stimulated Enzyme Production from C. versicolor in Shake Flask Cultures

The effects of different carbon sources on enzyme production were investigated: using glucose, crystalline cellulose powder or CMC, each at 2% w/v in the medium. The results are shown in Table II.

TABLE II
The effect of carbon source and inducer on the activities
of enzymes cultured from C. versicolor.

| | Enzyme | Media | |
|---|---|---|---|
| | activities | composition | Enzyme |
| | Cellulase | Xylanase | activities |
| | (μmol glucose | (μmol glucose | Laccase |
| Media | released per ml | equivalent released | (ΔOD₄₄₀ per ml |
| composition | per h) | per ml per h) | per min) |
| 2% CMC | 0.72 | 1.02 | 0.20 |
| 2% CMC + | 1.58 | 1.57 | 0.62 |
| 1% inducer | | | |
| 2% cellulose | 0.80 | 1.22 | 0.22 |
| 2% cellulose + | 0.95 | 1.21 | 0.32 |
| 1% inducer | | | |
| 2% CMC + | 1.64 | 0.95 | 0.78 |
| 0.5% glucose + | | | |
| 1% inducer | | | |
| 2% CMC + | 1.53 | 0.89 | 1.8 |
| 1% glucose + | | | |
| 1% inducer | | | |

From these tests, CMC is the preferred carbon source for production of all three enzymes. Addition of 0.5-1.0% inducer at day 3 of growth stimulated enzyme production by 80-100% from day 6 for xylanase and laccase and from day 8 for cellulase. Addition of different concentrations of CMC was investigated (0.5 to 2%) for the effect on enzyme production: all enzyme activities increased as the concentration of CMC was increased in the medium, up to 2% CMC.

The optimum conditions for simultaneous production of cellulase, xylanase and laccase activities in shake flasks were 2% w/v CMC in the medium as carbon source, 0.5-1.0% w/v inducer added at day 3 of growth. The cultures reached the optimum enzyme activities at 8 days growth, with approximately equal cellulase and xylanase activities. For application to treating dirty cattle hides, GB 2,325,241 indicates the preferred ratio of cellulase to xylanase as 2:1, together with good laccase activity.

If 1% glucose was added to the CMC medium, the titre for laccase activity was increased substantially, three fold, with cellulase unchanged, but xylanase titre reduced by 30%, see Table IV. This resulted in a cellulase to xylanase ratio closer to 2:1, but with substantially increased laccase activity, which meets the preferred mixture requirements more closely.

### EXAMPLE 3

### The Effect of Agitation on Shake Flask Cultures of C. versicolor

The agitation rate of the cultures (affecting availability of dissolved oxygen) was found to be critical in maximising enzyme production. At day 8 of growth, with the inducer added at day 3, cellulase, xylanase and laccase activities maintained higher levels when agitation was at 150 rpm, compared with 200 rpm. Optimum activities of all three enzymes occurred at day 8 under these conditions, as shown in FIGS. 2a, 2b and 2c comparing shaker speeds in enzyme production.

### EXAMPLE 4

### Simultaneous Production of Cellulase, Xylanase and Laccase by Culturing C. versicolor in 2 Litre Bioreactors

The medium optimised for shake flasks, using 2% CMC as carbon source and addition of inducer on day 3 of fungal growth, was used in the bioreactor, with agitation at 150 rpm. FIGS. 3a, 3b and 3c show enzyme activities from typical fermentation, with maximum activities occurring from day 5 to day 10 of growth for cellulase and xylanase and at day 8 for laccase. Dissolved oxygen concentration was maintained between 20 and 100% throughout the fermentation,

### EXAMPLE 5

### Simultaneous Production of Cellulase, Xylanase and Laccase by Culturing C. versicolor in 20 Litre and 75 Litre Bioreactors

The same conditions as described for a 2 litre bioreactor, given in Example 4, were used for growing the fungus in a 20 litre bioreactor. Dissolved oxygen concentration was maintained at 40 to 100%. It was observed that maximum cellulase, xylanase and laccase production was obtained between days 6 and 12 of growth.

Using a 75 litre bioreactor, similar enzyme titres were obtained, providing the dissolved oxygen concentration did not fall below 30%.

### EXAMPLE 6

### Stability of the Enzyme Mixture

Enzymes in the culture fluid, harvested on day 8 of growth, were concentrated through a membrane concentrator with a cut off at 10,000 Daltons, then the concentrate was freeze dried.

The powder was stored at room temperature, 4° C. and −20° C. and the activity was assayed over a three month period. Laccase activity disappeared after 3 months at room temperature and reduced by 50% at −20° C. Cellulase and xylanase activities had not decreased after three months at −20° C. or 4° C., but a slight reduction was observed in xylanase activity after storage at room temperature.

### EXAMPLE 7

### Preparation of Dung-Extract for Use as Inducer

The procedures described in the preceding Examples use dried sterilised cow dung as inducer. Handling of the inducer and the accuracy of measurement is improved by use of a liquid extract of dung as the inducer. An aqueous extract is prepared as follows.

To two parts by volume of dung is added one part by volume of water. The mixture is heated to 70° C., under constant mixing, and then held at 70° C. for 1 hour under constant mixing. After cooling, the mixture is filtered through muslin, allowing tiny particles to pass through. The thus obtained liquid dung extract is sterilised by autoclaving at 121° C. for 30 minutes, allowed to cool and stored at room temperature until required.

### EXAMPLE 8

### Removing Dung from Walls of Dairy Parlours and Farm Buildings

Dung-clad walls in a dairy parlour were sprayed with water to moisten the dung, then sprayed with an aqueous solution of a crude mixture of the enzymes, cellulase, xylanase and laccase, obtained by cultivation as in Example 5. After 60 min the walls were resprayed with water from a medium pressure hose, which removed the softened and degraded dung. The effect of the enzyme mixture is demonstrated in FIGS. 4 a and 4 b which show respectively photographs of a concrete wall before and after treatment.

Dung-spattered doors of farm buildings were similarly treated. The effect of the enzyme mixture is demonstrated in FIGS. 4 c and 4 d which show respectively photographs of a painted door before and after treatment.

### EXAMPLE 9

### Degradation of Pigeon Droppings (Pigeon Guano)

The basic composition of pigeon droppings is shown in the table below

| Components | Content on dry weight (%) |
|---|---|
| Cellulose/hemicellulose | 18 |
| Lignin | 36 |
| Uric acid | 24 |
| Water soluble components | 22 |

The uric acid content of pigeon guano can be degraded by a uricase enzyme; uric acid is broken down to allantoin. FIG. 5 shows the activity of uricase on pigeon guano.

The degradation of cellulose components in pigeon guano by lignocellulases and uricase was demonstrated as follows. The lignocellulase materal used was Klenzkin™—a mite of the enzymes, cellulase, xylanase and laccase in the approximate proportions 1:1.5:3 of laccase:cellulase:xylanase available from Klenzyme Ltd, and prepared by cultivation as in Example 5

Firstly 0.1 g of finely ground pigeon droppings in 5 ml of phosphate buffer at pH 6.5 was incubated at 37° C. for 2 hours.

Then addition of enzymes:
Klenzkin™ (0.2 ml added from a stock solution of 0.11 g/ml).
Uricase (0.2 ml added from a stock solution of 0.826 units/ml)
   The solution was incubated again for 1 hour at 37° C., then analysed.

Measurement of reducing sugars released from cellulosic components by the enzymes is shown in FIG. 6. Klenzkin (lignocellulase enzymes) degraded cellulose in the droppings whereas uricase had no effect on cellulose degradation.

Accordingly, uricase alone will not satisfactorily degrade pigeon droppings for removal in a cleaning process. However the use of a blend of uricase and a mixture of cellulase, xylanase and laccase, results in degradation of the main components of pigeon droppings so that the droppings are readily removed by a cleaning process as in Example 7.

1. A method for the degradation of lignocellulosic material by applying to the material an enzyme composition which is a mixture comprising at least a cellulase, xylanase and ligninase, and optionally other enzymes, to solubilise or decompose the material at least partially.

2. A method according to claim 1 in which the enzyme composition further includes a protease, lipase, urease, uricase, and/or pectinase.

3. A method of removing a biological deposit from a surface or location on or in which it is undesirably deposited, by applying to the deposit an enzyme composition which is a mixture comprising at least a cellulase, xylanase and ligninase to solubilise or decompose the deposit.

4. A method according to claim 3 in which the deposit is human or animal faeces and the enzyme composition comprises a protease, lipase, urease, cellulase, xylanase and ligninase.

5. A method according to claim 3 in which the deposit is bird droppings and the enzyme composition comprises a uricase, cellulase, xylanase and ligninase.

6. A method according to claim 3 in which the deposit is leaves and the enzyme composition comprises a pectinase, cellulase, xylanase and ligninase

7. A method according to claim 1 in which the enzyme composition is a mechanical blend of the enzymes.

8. A method according to claim 1 in which the enzyme composition includes an enzyme mixture obtainable by cultivating a fungus selected from the class of White Rot Fungi in a liquid growth medium and harvesting the enzymes produced by the fungus from the liquid growth medium.

9. A method according to claim 8, in which the fungus is cultivated in the presence of dung or a dung extract as an auxiliary growth medium.

10. An enzyme composition useful to solubilise or decompose a biological deposit, which is a enzyme mixture comprising at least a cellulase, xylanase and ligninase, and at least one other enzyme selected from a protease, lipase, urease, uricase, and pectinase.

11. A composition according to claim 10 in which the deposit is human or animal faeces and the enzyme composition comprises a protease, lipase, urease, cellulase, xylanase and ligninase.

12. A composition according to claim 10 in which the deposit is bird droppings and the enzyme composition comprises a uricase, cellulase, xylanase and ligninase

13. A composition according to claim 10 in which the deposit is leaves and the enzyme composition comprises a pectinase, cellulase, xylanase and ligninase

14. A composition according to claim 10 in which the enzyme composition includes an enzyme mixture obtained by cultivating a fungus selected from the class of White Rot Fungi in a liquid growth medium and harvesting the enzymes produced by the fungus from the liquid growth medium.

15. A composition according to claim 14, in which the fungus is cultivated in the presence of dung or a dung extract as an auxiliary growth medium.

16. A composition according to claim 15 in which the fungus is selected from the family Polyporaceae.

17. A composition according to claim 15, in which the enzyme mixture includes cellulase, xylanase and laccase enzymes.

18. A composition according to claim 17 in which the fungus is selected from the species Coriolus, Pleurotus and Ganoderma.

19. A composition according to claim 16 in which the fungus is selected from Coriolus versicolor, Pleurotus ostreatus and Ganoderma applanatum.

20. A composition according to claim 15, in which the enzyme mixture includes cellulase, xylanase and lignin peroxidase enzymes.

21. A composition according to claim 20 in which the fungus is selected from the species Phanerochaete.

22. A composition according to claim 21 in which the fungus is Phanerochaete chrysosporium.

23. A composition according to any claim 14 in which, after a suitable growth period, residues are removed from the nutrient medium by filtration, and the enzyme mixture is harvested, and then dried.

24. A composition according to claim 23 in which the enzyme mixture is freeze-dried or spray-dried composition.
